(19) United States
(12) Patent Application Publication
(10) Pub. No.: US 2024/0199166 A1
(43) Pub. Date: Jun. 20, 2024

(54) CONTROL MODULE, HANDLEBAR ASSEMBLY WITH THE CONTROL MODULE, AND ELECTRIC ASSIST VEHICLE WITH THE HANDLEBAR ASSEMBLY

(71) Applicant: FOXCONN INTERCONNECT TECHNOLOGY LIMITED, Grand Cayman (KY)

(72) Inventor: CHIEN-WEN WANG, New Taipei (TW)

(21) Appl. No.: 18/207,637

(22) Filed: Jun. 8, 2023

(30) Foreign Application Priority Data
Dec. 16, 2022 (CN) 202211627870.2

(51) Int. Cl. B62M 6/50; A61B 5/00; B62J 6/028; B62J 43/20; B62J 43/30; B62J 45/20; B62J 45/416; G08C 17/02

(52) U.S. Cl. CPC B62M 6/50; A61B 5/0002; B62J 6/028; B62J 43/20; B62J 43/30; B62J 45/20; B62J 45/416; G08C 17/02; B62K 23/02

(57) ABSTRACT

A control module is provided. The control module is installed on a handlebar of an electric assist vehicle. The lighting unit, the wireless communication unit, the input unit, and the power management unit of the control module each is electrically coupled to the control unit of the control module. The control unit is configured to receive a first operation signal inputted via the input unit or from the wireless communication unit, and control a lighting mode of the lighting unit accordingly. The control unit is configured to receive a second operation signal inputted via the input unit or from the wireless communication unit, and output a motor control signal to the power management unit according to the second operation signal, the power management unit accordingly control an assisting mode of an assist power of a motor. A related handlebar assembly and a related electric assist vehicle are provided.

### FIELD

The subject matter herein generally relates to a mobile vehicle technology, and particularly to a control module, a handlebar assembly with the control module, and an electric assist vehicle with the handlebar assembly.

### BACKGROUND

To increase a driving safety at night or under a rain and fog weather, a lighting lamp may be needed to be installed on a vehicle such as an electric assist vehicle. However, nowadays, the lighting lamp is usually individually set on the electric assist vehicle, adding a cost of the lighting lamp to a cost of the electric assist vehicle. Further, a weight of the electric assist vehicle is increased, an occupied space in the electric assist vehicle is increased, and an aesthetic of the electric assist vehicle may be affected.

### SUMMARY

An embodiment of the present application provides a control module, a handlebar assembly with the control module, and an electric assist vehicle with the handlebar assembly which can decrease the cost of the electric assist vehicle, the weight of the electric assist vehicle, and the space occupied by the electric assist vehicle, and increase the aesthetic of the electric assist vehicle.

In a first aspect, an embodiment of the present application provides a control module. The control module is configured to be installed on a handlebar of an electric assist vehicle. The control module includes a lighting unit, a control unit, a wireless communication unit, an input unit, and a power management unit. The lighting unit, the wireless communication unit, the input unit, and the power management unit each is electrically coupled to the control unit. The wireless communication unit is configured to receive a wireless signal, convert the wireless signal to an operation signal, and output the operation signal to the control unit. The wireless communication unit is further configured to receive a control signal from the control unit, convert the control signal to a wireless signal, and emit the wireless signal outward. The control unit is configured to receive a first operation signal inputted via the input unit or receive the first operation signal from the wireless communication unit, and control a lighting mode of the lighting unit according to the first operation signal. The control unit is configured to receive a second operation signal inputted via the input unit or receive the second operation signal from the wireless communication unit, and output a motor control signal according to the second operation signal. The power management unit is configured to receive the motor control signal, and control an assisting mode of an assist power of a motor according to the motor control signal.

According to some embodiments of the present application, the lighting mode of the lighting unit includes a low-beam lamp pattern, a high-beam lamp pattern, a blinking pattern, and a non-lighting pattern. The assisting mode of the assist power of the motor includes an increasing mode of the assist power of the motor and a decreasing mode of the assist power of the motor.

According to some embodiments of the present application, the lighting unit is on a first side of the control module, and the lighting unit is configured to emit a lighting outward. The input unit is on a second side of the control module, and the input unit is accessible. A surface of the second side is coupled to a surface of the first side.

According to some embodiments of the present application, the control module further includes a battery compartment and a side cover. The battery compartment and the side cover are on a third side of the control module, the third side of the control module is opposite to the first side of the control module, the battery compartment is configured to receive the battery, and the side cover rotatably covers the battery compartment.

According to some embodiments of the present application, the control module defines an installing hole at a fourth side of the control module, the fourth side is opposite to the second side. The control module further defines a slot at a surface of the fourth side, the slot communicates with the installing hole, the installing hole is configured to receive the handlebar via the slot.

According to some embodiments of the present application, the control unit controls the lighting unit to become a blinking pattern when the control unit of the control module receives a calling signal from an electronic device external to the control module.

In a second aspect, an embodiment of the present application provides a handlebar assembly. The handlebar assembly includes a handlebar and a control module. The control module is installed on the handlebar of an electric assist vehicle. The control module includes a lighting unit, a control unit, a wireless communication unit, an input unit, and a power management unit. The lighting unit, the wireless communication unit, the input unit, and the power management unit each is electrically coupled to the control unit. The wireless communication unit is configured to receive a wireless signal, convert the wireless signal to an operation signal, and output the operation signal to the control unit. The wireless communication unit is further configured to receive a control signal from the control unit, convert the control signal to a wireless signal, and emit the wireless signal outward. The control unit is configured to receive a first operation signal inputted via the input unit or receive the first operation signal from the wireless communication unit, and control a lighting mode of the lighting unit according to the first operation signal. The control unit is configured to receive a second operation signal inputted via the input unit or receive the second operation signal from the wireless communication unit, and output a motor control signal according to the second operation signal. The power management unit is configured to receive the motor control signal, and control an assisting mode of an assist power of a motor according to the motor control signal.

In a third aspect, an embodiment of the present application also provides an electric assist vehicle. The electric assist vehicle includes a handlebar assembly. The handlebar assembly includes a handlebar and a control module. The control module is installed on the handlebar. The control module includes a lighting unit, a control unit, a wireless communication unit, an input unit, and a power management unit. The lighting unit, the wireless communication unit, the input unit, and the power management unit each is electrically coupled to the control unit. The wireless communication unit is configured to receive a wireless signal, convert the wireless signal to an operation signal, and output the operation signal to the control unit. The wireless communication unit is further configured to receive a control signal from the control unit, convert the control signal to a wireless signal, and emit the wireless signal outward. The control unit is configured to receive a first operation signal inputted via the input unit or receive the first operation signal from the wireless communication unit, and control a lighting mode of the lighting unit according to the first operation signal. The control unit is configured to receive a second operation signal inputted via the input unit or receive the second operation signal from the wireless communication unit, and output a motor control signal according to the second operation signal. The power management unit is configured to receive the motor control signal, and control an assisting mode of an assist power of a motor according to the motor control signal.

The disclosure can integrate the lighting unit, the control unit, the wireless communication unit, the input unit, the power management unit, and the energy management unit to the handlebar, to increase an aesthetics of the electric assist vehicle, and decrease the number of the components of the electric assist vehicle, thus a development costs of a mold is lower, and a weight of the electric assist vehicle is decreased.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a schematic view of an embodiment of an electric assist vehicle.

FIG. 2 is a block diagram of an embodiment of a control module of the electric assist vehicle of the FIG. 1

FIG. 3 is a schematic view of an embodiment showing a control module installed on a handlebar.

FIG. 4A is a front view of an embodiment of a control module of the FIG. 3.

FIG. 4B is a top view of an embodiment of the control module of the FIG. 3

FIG. 4C is a side view of an embodiment of the control module of the FIG. 3.

FIG. 5 is a schematic view of another embodiment of a handlebar assembly.

FIG. 6A is a side view of an embodiment of the handlebar assembly of the FIG. 5.

FIG. 6B is a partial front view of an embodiment of the handlebar assembly of the FIG. 5.

FIG. 6C is a partial rear view of an embodiment of the handlebar assembly of the FIG. 5.

### DETAILED DESCRIPTION

For clarity, of illustration of objectives, features and advantages of the present disclosure, the drawings combined with the detailed description illustrate the embodiments of the present disclosure hereinafter. It is noted that embodiments of the present disclosure and features of the embodiments can be combined, when there is no conflict.

Various details are described in the following descriptions for better understanding of the present disclosure, however, the present disclosure may also be implemented in other ways other than those described herein. The scope of the present disclosure is not to be limited by the specific embodiments disclosed below.

Unless defined otherwise, all technical and scientific terms used herein have the same meaning as commonly understood by one of ordinary skill in the art to which the present disclosure belongs. The terms used herein in the present disclosure are only for the purpose of describing specific embodiments, and are not intended to limit the present disclosure.

To increase a driving safety at night or under a rain and fog weather, a lighting lamp may be needed to be installed on a vehicle such as an electric assist vehicle. However, nowadays, the lighting lamp is usually individual set on the electric assist vehicle, causing a cost of the lighting lamp to be added into a cost of the electric assist vehicle, which may increase the cost of the electric assist vehicle. Further, a weight of the electric assist vehicle is increased, a space occupied by the electric assist vehicle is increased, and an aesthetic of the electric assist vehicle may be influenced.

An embodiment of the disclosure provides a control module 80. The control module 80 can be installed on an electric assist vehicle 100 to control the electric assist vehicle 100. The electric assist vehicle 100 can be an electric assist bicycle, an electric assist tricycle, or the like. The electric assist vehicle 100 can be any one of an electric vehicle and a hybrid vehicle. For the convenience of description, the electric assist bicycle can be used as an example to illustrate the electric assist vehicle 100.

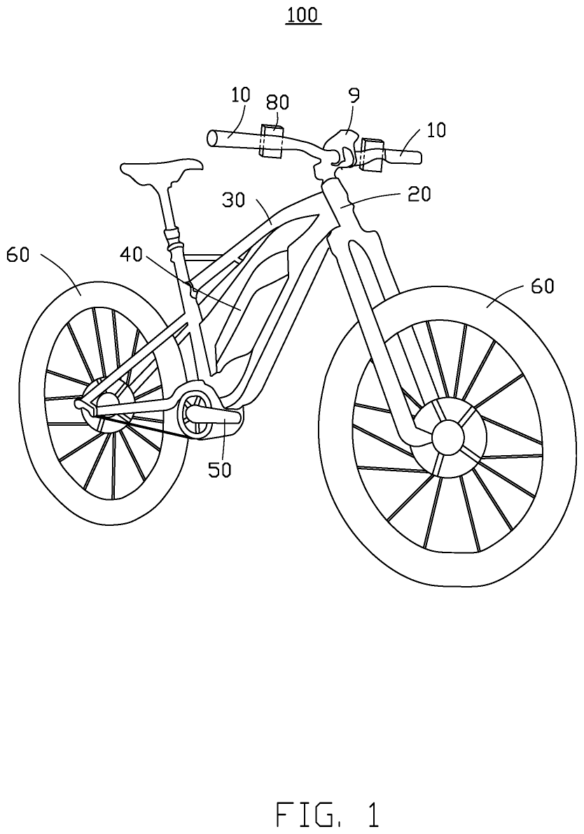

Referring to FIG. 1, the electric assist vehicle 100 includes two handlebars 10, a front frame 20, a vehicle frame 30, an energy storage device 40, a motor 50, and two wheels 60. Two handlebars 10 are respectively arranged at two ends of the front frame 20 of the electric assist vehicle 100. The energy storage device 40 is installed on the vehicle frame 30. The energy storage device 40 is configured to provide power for the motor 50. The motor 50 is configured to convert an electric power to a mechanical power, to drive the wheels 60 of the electric assist vehicle 100 to rotate, thereby driving the electric assist vehicle 100 to move.

In some embodiments, the control module 80 is installed on one handlebar 10 of the electric assist vehicle 100, thus it is convenient for a user to operate.

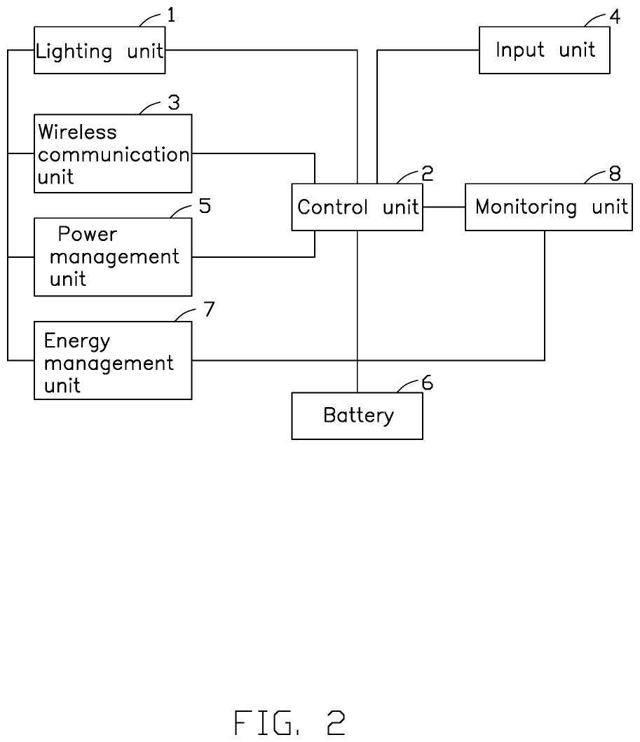

Referring also to FIGS. 1-2, in the embodiment, the control module 80 includes a lighting unit 1, a control unit 2, a wireless communication unit 3, an input unit 4, and a power management unit 5. The lighting unit 1, the wireless communication unit 3, the input unit 4, and the power management unit 5 each is electrically coupled to the control unit 2.

The wireless communication unit 3 can be configured to receive a wireless signal, convert the wireless signal to an operation signal, and output the operation signal to the control unit 2. The wireless communication unit 3 is further configured to receive a control signal from the control unit 2, convert the control signal to a wireless signal, and emit the wireless signal outward. The control unit 2 can receive a first operation signal inputted via the input unit 4 or receive the first operation signal from the wireless communication unit 3, and control a lighting mode of the lighting unit 1 according to the first operation signal. The lighting mode of the lighting unit 1 can include a low-beam lamp pattern, a high-beam lamp pattern, a blinking pattern, and a non-lighting pattern. The control unit 2 can receive a second operation signal inputted via the input unit 4 or receive the second operation signal from the wireless communication unit 3, and output a motor control signal according to the second operation signal. The motor control signal can be a power control signal. The power management unit 5 is configured to receive the motor control signal, and control an assisting mode of an assist power of the motor 50 according to the motor control signal. The assisting mode of the assist power of the motor 50 can include an increasing mode of the assist power of the motor 50 and a decreasing mode of the assist power of the motor 50. In the increasing mode of the assist power of the motor 50, the power management unit 5 can control the motor 50 to increase the assist power. In the decreasing mode of the assist power of the motor 50, the power management unit 5 can control the motor 50 to decrease the assist power.

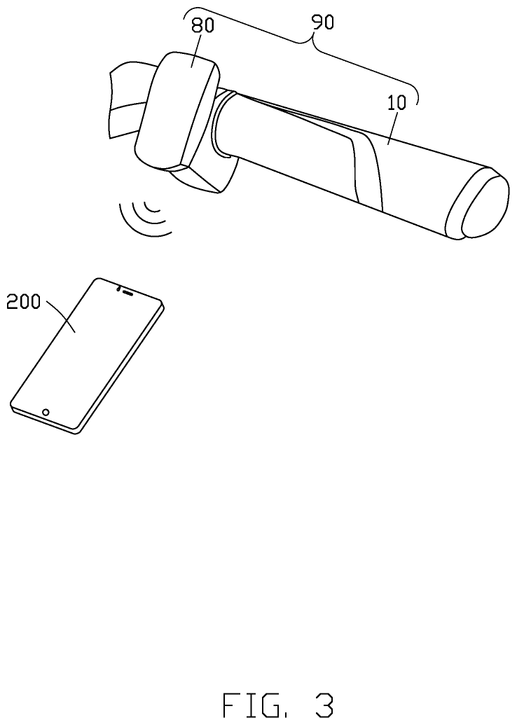

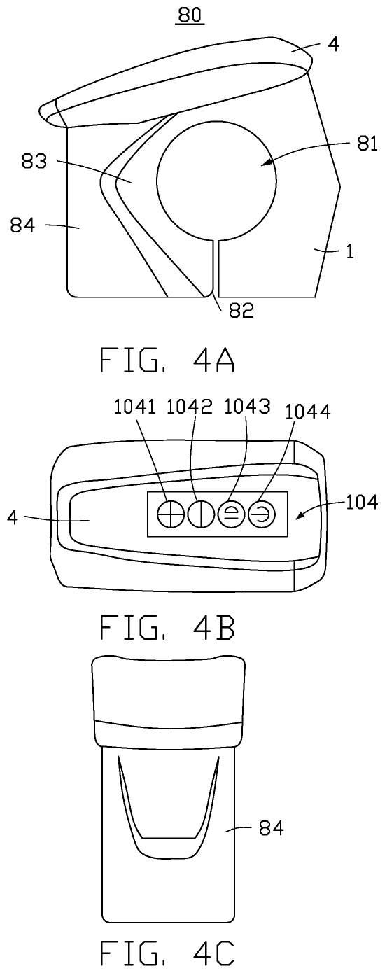

Referring also to FIG. 3, in the embodiment, a shape of the control module 80 can be substantial cuboid. The control module 80 can be detachably installed on the handlebar 10. The control module 80 further defines an installing hole 81 (referring to FIG. 4A) at a lower end of the control module 80, and further defines a slot 82 at a surface of the lower end. The slot 82 communicates with the installing hole 81. Thus, the control module 80 can be slight deformed via the slot 82 to cause the installing hole 81 to receive the handlebar 10. And, the control module 80 can be accordingly installed on the handlebar 10. In other embodiments, the control module 80 can be coupled to the handlebar 10 via a connecting member such as a hook, or can be adhered to the handlebar 10.

Referring to FIG. 4A, the lighting unit 1 is on a side of the control module 80, to emit a lighting outward. In some embodiments, the lighting unit 1 is on a first side of the control module 80.

Referring to FIGS. 1-2, 4A, and 4B, the input unit 4 is on a second side of the control module 80. A surface of the second side is coupled to a surface of the first side. In some embodiments, the input unit 4 can be a number of buttons 104. Each button 104 can be operated by a user to perform a corresponding function. In the embodiments, each button 104 can be a physical button. The buttons 104 can include a first button 1041, a second button 1042, a third button 1043, and a fourth button 1044. The first button 1041 and the second button 1042 each is configured to trigger a control of the assist power of the motor 50 via the power management unit 5. For example, in some embodiments, when the first button 1041 is pressed, the control unit 2 can receive an operation signal of pressing the first button 1041, and output a motor control signal for increasing the assist power of the motor 50 to the power management unit 5 according to the operation signal of pressing the first button 1041. The power management unit 5 can control the motor 50 to increase the assist power according to the motor control signal for increasing the assist power of the motor 50. For example, in some embodiments, when the second button 1042 is pressed, the control unit 2 can receive an operation signal of pressing the second button 1042, and output a motor control signal for decreasing the assist power of the motor 50 to the power management unit 5 according to the operation signal of pressing the second button 1042. The power management unit 5 can control the motor 50 to decrease the assist power according to the motor control signal for decreasing the assist power of the motor 50.

The third button 1043 can be configured to trigger a control of the lighting mode of the lighting unit 1. For example, in some embodiments, when the third button 1043 is pressed at a first time, the control unit 2 can receive an operation signal of first pressing of the third button 1043, and control the lighting unit 1 to become a first lighting mode according to the operation of first pressing of the third button 1043. The first lighting mode can be, for example the low-beam lamp pattern. For example, in some embodiments, when the third button 1043 is pressed at a second time, the control unit 2 can receive an operation signal of second pressing of the third button 1043, and control the lighting unit 1 to become a second lighting mode according to the operation of second pressing of the third button 1043. The second lighting mode can be, for example the high-beam lamp pattern. For example, in some embodiments, when the third button 1043 is pressed at a third time, the control unit 2 can receive an operation signal of third pressing of the third button 1043, and control the lighting unit 1 to become a third lighting mode according to the operation of third pressing of the third button 1043. The third lighting mode can be, for example the blinking pattern. For example, in some embodiments, when the third button 1043 is pressed at a fourth time, the control unit 2 can receive an operation signal of fourth pressing of the third button 1043, and control the lighting unit 1 to become a fourth lighting mode according to the operation of fourth pressing of the third button 1043. The fourth lighting mode can be, for example the non-lighting pattern. In the fourth lighting mode, the lighting unit 1 is turned off, and stop the lighting.

The fourth button 1044 can be configured to trigger a starting upon power-on of the control unit 2 of the control module 80. It can be understood that, in some embodiments, the control module 80 further includes a battery 6 and an energy management unit 7. The energy management unit 7 is electrically coupled to the battery 6. The energy management unit 7 is further electrically coupled to some power consuming units, for example the lighting unit 1, the control unit 2, the wireless communication unit 3, the power management unit 5, and so on. The energy management unit 7 can be configured to receive power from the battery 6, and provide the power to the power consuming units of the control module 80 via the control unit 2. The energy management unit 7 can be further configured to charge the battery 6 via the control unit 2. In some embodiments, for example, when the fourth button 1044 is pressed, the energy management unit 7 can receive the power from the battery 6 and provide the power to the control unit 2. After the control unit 2 is started, the control unit 2 can control the power management unit 5 to step up or step down the power from the battery 6, to provide different powers to different power consuming units.

It can be understood that, the input unit 4 can be a display device, and each button can be a virtual button displayed on the display device, the disclosure is not limited herein.

It can be understood that, the number of the buttons, the type of the buttons, and the function of the buttons can be various, for example, in the other embodiments, the buttons can include one or more physical buttons and one or more virtual buttons, and the disclosure is not limited herein.

Referring to FIGS. 3, 4A, and 4C, in some embodiments, the control module 80 further includes a battery compartment 83 and a side cover 84. The battery compartment 83 and the side cover 84 are arranged on a third side of the control module 80. The third side of the control module 80 is opposite to the first side of the control module 80. The side cover 84 rotatably covers the battery compartment 83. The battery compartment 83 is configured to receive the battery 6. In some embodiments, the battery 6 can be a detachable battery. Thus, the side cover 84 can be rotate to be open, and the battery 6 in the battery compartment 83 can be replaced. In some embodiments, the battery 6 can be a rechargeable battery. Thus, the control module 80 can be taken away from the handlebar 10, and be charged to charge the battery 6.

The slot 82 is defined in a fourth side of the control module 80. The fourth side of the control module 80 is opposite to the second side of the control module 80.

In the embodiment, when the control module 80 is installed on the handlebar 10, the input unit 4 faces toward the user, thus the user can operation the input unit 4 of the control module 80 to control the electric assist vehicle at any time. The lighting unit 1 faces toward a front of the electric assist vehicle, to light a road for the user. Thus, the user can know a condition of the road. The side cover 84 faces toward a rear of the electric assist vehicle. The control unit 2, the wireless communication unit 3, and the power management unit 5 each is an inner of the control module 80.

Referring back to FIGS. 1-2, in some embodiments, a monitoring unit 8 is arranged on a portion of the handlebar 10 which is far away from the input unit 4 of the control module 80. In the embodiment, the monitoring unit 8 is arranged on a lower portion of the handlebar 10 away from the input unit 4. The monitoring unit 8 can be touched by the hand of the user. The monitoring unit 8 can be configured to detect heart beat rate data and/or blood oxygen saturation data of the user. The monitoring unit 8 can communicate with the control unit 2 of the control module 80 via a wireless connection or a wired connection. The control unit 2 can obtain the heart beat rate data and/or the blood oxygen saturation data of the user from the monitoring unit 8, to monitor a health of the user.

In some embodiments, the electric assist vehicle 100 further includes a display unit 9. The display unit 9 is installed on the front frame 20, and is arranged between the two handlebars 10. The display unit 9 is configured to display data. The display unit 9 is electrically coupled to the control unit 2 of the control module 80. The control unit 2 can control the display unit 9 to display various data of the electric assist vehicle 100, for example, a battery power, a mileage, a lighting mode, a current range, and so on.

In some embodiments, the control unit 2 can communicate with the motor 50 via the wireless communication unit 3. The control unit 2 can receive information from the motor 50 via the wireless communication unit 3, and control the motor 50 to rotate via the wireless communication unit 3 and the power management unit 5.

In some embodiments, referring also to FIG. 3, the control unit 2 of the control module 80 can further communicate with an electronic device 200 via the wireless communication unit 3. The electronic device 200 can be, for example a mobile phone. The control unit 2 can transmit the various data of the electric assist vehicle 100, and the heart beat rate data and/or the blood oxygen saturation data of the user to the mobile phone. In the embodiment, the wireless communication unit 3 can communicate with the electronic device 200 or the electric assist vehicle 100 via at least one of a group consisting of a Bluetooth manner, an ANT+ manner, a Zigbee manner, an UWB manner, and so on.

In some embodiments, when the control unit 2 of the control module 80 receives a calling signal from the electronic device 200, the control unit 2 can control the lighting unit 1 to become the third lighting mode. Thus, the lighting unit 1 can continue to blink, and the user can quickly find the electric assist vehicle 100 at a stop point via a blink of the lighting unit 1 of the electric assist vehicle 100.

In some embodiments, the electric assist vehicle 100 can include two control modules 80. Two control modules 80 are respectively arranged on two handlebars 10 of the electric assist vehicle 100. Thus, the user can control the electric assist vehicle 100 conveniently via the control modules 80 on the two handlebars 10.

It can be understood that, each control module 80 and a corresponding handlebar 10 can form a handlebar assembly 90, the disclosure is not limited herein.

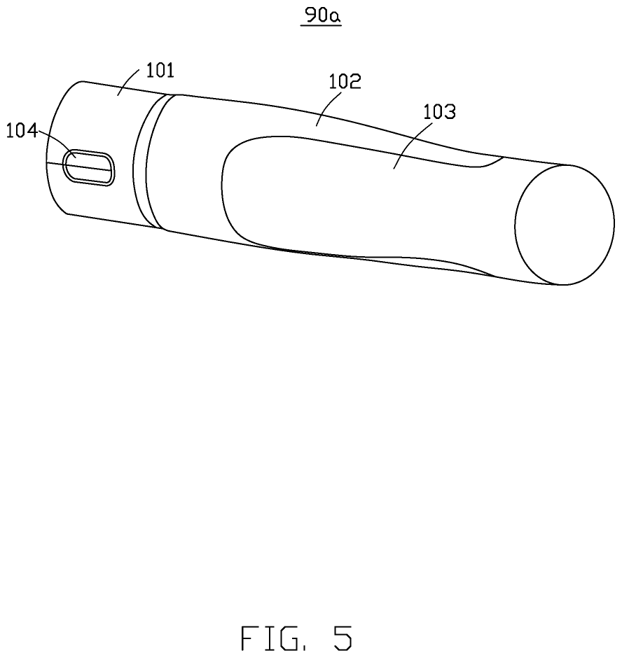

Referring to FIGS. 2 and 5, in other embodiments, the lighting unit 1, the control unit 2, the wireless communication unit 3, the power management unit 5, the energy management unit 7, and the monitoring unit 8 of the FIG. 2 can be integrated into the handlebar 10 of the electric assist vehicle 100.

For example, another handlebar assembly 90a is provided. In some embodiments, a shape of the handlebar assembly 90a can be substantial cylindrical. The handlebar assembly 90a includes a first casing 101 and a second casing 102. The first casing 101 is coupled to the second casing 102. In the embodiment, a first end of the first casing 101 is coupled to the front frame 20, and a second end of the first casing 101 which is away from the front frame is coupled to the second casing 102. The handlebar assembly 90a further includes an anti-slip socket 103. The anti-slip socket 103 is sleeved on a surface of the second casing 102. It can be understood that, during travelling, a user can grasp the anti-slip socket 103, the disclosure is not limited herein.

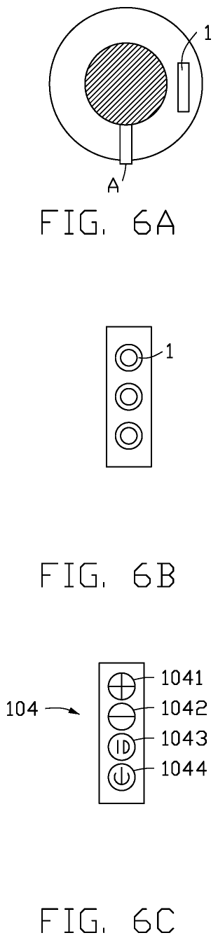

Referring also to FIGS. 6A-6C, a side view of the handlebar assembly 90a, a partial front view of the handlebar assembly 90a, and a partial rear view of the handlebar assembly 90a are shown. In the FIGS. 6A-6C, the lighting unit 1 is fit onto a side of the handlebar assembly 90a which faces toward a front of the electric assist vehicle. Thus, when the lighting unit 1 lights under a control of the control unit 2, the lighting unit 1 can light the road in front of the electric assist vehicle for the user.

The control unit 2, the wireless communication unit 3, the power management unit 5, and the energy management unit 7 are arranged in the handlebar assembly 90a. In the embodiment, the control unit 2, the wireless communication unit 3, the power management unit 5, and the energy management unit 7 can be arranged in the first casing 101 and/or the second casing 102. The control unit 2 can be electrically coupled to the battery 6 and the motor via a wire, and the power management unit 5 can be electrically coupled to the motor via a wire. In the embodiment, a lower side of the handlebar assembly 90a further define an open A extending along a radial direction of the handlebar assembly 90a. It can be understood that, the wire can be coupled to the control unit 2 and the power management unit 5, and can extend out from the handlebar 10 via the open A to be coupled to the energy storage device 40 and the motor 50. In the embodiment, the buttons are arranged on a side of the handlebar assembly 90a which faces toward a rear of the electric assist vehicle. The monitoring unit is arranged on a portion of the handlebar 10 near the anti-slip socket 103. In the embodiment, the monitoring unit is arranged on a lower portion of the handlebar 10 below the anti-slip socket 103. Thus, it is convenient for the hand of the user to touch the monitoring unit. The monitoring unit can be configured to detect the heart beat rate data and/or the blood oxygen saturation data of the user. Thus, the disclosure can integrate the lighting unit 1, the control unit 2, the wireless communication unit 3, the input unit 4, the power management unit 5, and the energy management unit 7 to the handlebar 10, to increase an aesthetics of the electric assist vehicle, and decrease the number of the components of the electric assist vehicle, thus a development costs of a mold is lower, and a weight of the electric assist vehicle is decreased.

In the embodiment, the lighting unit 1, the control unit 2, the wireless communication unit 3, the input unit 4, and the power management unit 5 are integrated to the handlebar 10, which can decrease the space occupied by the electric assist vehicle, and low a cost of the electric assist vehicle.

The handlebar assembly 90a of the embodiment further includes a monitoring unit. The monitoring unit can monitor a health of the user, to increase a driving safety.

The handlebar assembly 90a of the embodiment further includes a wireless communication unit 3. The wireless communication unit 3 can communicate with the electronic device. The handlebar assembly 90a can control the lighting unit 1 to blink when the handlebar assembly 90a receives a calling signal from the electronic device. Thus, the user can quickly find the electric assist vehicle at a stop point via a blink of the light unit of the electric assist vehicle.

It should be emphasized that the above-described embodiments of the present disclosure, including any particular embodiments, are merely possible examples of implementations, set forth for a clear understanding of the principles of the disclosure. Many variations and modifications can be made to the above-described embodiment(s) of the disclosure without departing substantially from the spirit and principles of the disclosure. All such modifications and variations are intended to be included herein within the scope of this disclosure and protected by the following claims.

What is claimed is:

1. A control module configured to be installed on a handlebar of an electric assist vehicle, the control module comprising a lighting unit, a control unit, a wireless communication unit, an input unit, and a power management unit, wherein:
the lighting unit, the wireless communication unit, the input unit, and the power management unit each is electrically coupled to the control unit,
the wireless communication unit is configured to receive a wireless signal, convert the wireless signal to an operation signal, and output the operation signal to the control unit; the wireless communication unit is further configured to receive a control signal from the control unit, convert the control signal to a wireless signal, and emit the wireless signal outward,
the control unit is configured to receive a first operation signal inputted via the input unit or receive the first operation signal from the wireless communication unit, and control a lighting mode of the lighting unit according to the first operation signal,
the control unit is configured to receive a second operation signal inputted via the input unit or receive the second operation signal from the wireless communication unit, and output a motor control signal according to the second operation signal, and
the power management unit is configured to receive the motor control signal, and control an assisting mode of an assist power of a motor according to the motor control signal.

2. The control module according to claim 1, wherein:
the lighting mode of the lighting unit comprises a low-beam lamp pattern, a high-beam lamp pattern, a blinking pattern, and a non-lighting pattern;
the assisting mode of the assist power of the motor comprises an increasing mode of the assist power of the motor and a decreasing mode of the assist power of the motor.

3. The control module according to claim 2, wherein:
the lighting unit is on a first side of the control module, and the lighting unit is configured to emit a lighting outward;
the input unit is on a second side of the control module, and the input unit is accessible;
a surface of the second side is coupled to a surface of the first side.

4. The control module according to claim 3 further comprising a battery compartment and a side cover, wherein:
the battery compartment and the side cover are on a third side of the control module, the third side of the control module is opposite to the first side of the control module, the battery compartment is configured to receive the battery, and the side cover rotatably covers the battery compartment.

5. The control module according to claim 4 further defining an installing hole at a fourth side of the control module, the fourth side being opposite to the second side; and further defining a slot at a surface of the fourth side, wherein the slot communicates with the installing hole, the installing hole is configured to receive the handlebar via the slot.

6. The control module according to claim 1, wherein:
the control unit controls the lighting unit to become a blinking pattern when the control unit of the control module receives a calling signal from an electronic device external to the control module.

7. A handlebar assembly comprising a handlebar and a control module, the control module being installed on the handlebar of an electric assist vehicle, the control module comprising a lighting unit, a control unit, a wireless communication unit, an input unit, and a power management unit, wherein:
the lighting unit, the wireless communication unit, the input unit, and the power management unit each is electrically coupled to the control unit,
the wireless communication unit is configured to receive a wireless signal, convert the wireless signal to an operation signal, and output the operation signal to the control unit; the wireless communication unit is further configured to receive a control signal from the control unit, convert the control signal to a wireless signal, and emit the wireless signal outward,
the control unit is configured to receive a first operation signal inputted via the input unit or receive the first operation signal from the wireless communication unit, and control a lighting mode of the lighting unit according to the first operation signal,
the control unit is configured to receive a second operation signal inputted via the input unit or receive the second operation signal from the wireless communication unit, and output a motor control signal according to the second operation signal, and
the power management unit is configured to receive the motor control signal, and control an assisting mode of an assist power of a motor according to the motor control signal.

8. The handlebar assembly according to claim 7, wherein:
the lighting mode of the lighting unit comprises a low-beam lamp pattern, a high-beam lamp pattern, a blinking pattern, and a non-lighting pattern;
the assisting mode of the assist power of the motor comprises an increasing mode of the assist power of the motor and a decreasing mode of the assist power of the motor.

9. The handlebar assembly according to claim 8, wherein:
the lighting unit is on a first side of the control module, and the lighting unit is configured to emit a lighting outward;
the input unit is on a second side of the control module, and the input unit is accessible;
a surface of the second side is coupled to a surface of the first side.

10. The handlebar assembly according to claim 9, wherein:
the control module further comprises a battery compartment and a side cover;
the battery compartment and the side cover are on a third side of the control module, the third side of the control module is opposite to the first side of the control module, the battery compartment is configured to receive the battery, and the side cover rotatably covers the battery compartment.

11. The handlebar assembly according to claim 10, wherein:
the control module defines an installing hole at a fourth side of the control module, the fourth side is opposite to the second side;
the control module further defines a slot at a surface of the fourth side, the slot communicates with the installing hole, the installing hole is configured to receive the handlebar via the slot.

12. The handlebar assembly according to claim 7, wherein:
the control unit controls the lighting unit to become a blinking pattern when the control unit of the control module receives a calling signal from an electronic device external to the control module.

13. The handlebar assembly according to claim 7, wherein:
the handlebar assembly further comprises a monitoring unit, the monitoring unit is arranged on a portion of the handlebar away from the input unit, the monitoring unit is configured to detect heart beat rate data and/or blood oxygen saturation data of a user, the monitoring unit communicates with the control unit of the control module via a wireless connection or a wired connection;
the control unit is configured to obtain the heart beat rate data and/or the blood oxygen saturation data of the user from the monitoring unit, to monitor a health of the user.

14. A electric assist vehicle comprising a handlebar assembly, the handlebar assembly comprising a handlebar and a control module, the control module being installed on the handlebar, the control module comprising a lighting unit, a control unit, a wireless communication unit, an input unit, and a power management unit, wherein:
the lighting unit, the wireless communication unit, the input unit, and the power management unit each is electrically coupled to the control unit,
the wireless communication unit is configured to receive a wireless signal, convert the wireless signal to an operation signal, and output the operation signal to the control unit; the wireless communication unit is further configured to receive a control signal from the control unit, convert the control signal to a wireless signal, and emit the wireless signal outward,
the control unit is configured to receive a first operation signal inputted via the input unit or receive the first operation signal from the wireless communication unit, and control a lighting mode of the lighting unit according to the first operation signal,
the control unit is configured to receive a second operation signal inputted via the input unit or receive the second operation signal from the wireless communication unit, and output a motor control signal according to the second operation signal, and
the power management unit is configured to receive the motor control signal, and control an assisting mode of an assist power of a motor according to the motor control signal.

15. The electric assist vehicle according to claim 14, wherein:
the lighting mode of the lighting unit comprises a low-beam lamp pattern, a high-beam lamp pattern, a blinking pattern, and a non-lighting pattern;
the assisting mode of the assist power of the motor comprises an increasing mode of the assist power of the motor and a decreasing mode of the assist power of the motor.

16. The electric assist vehicle according to claim 15, wherein:
the lighting unit is on a first side of the control module, and the lighting unit is configured to emit a lighting outward;
the input unit is on a second side of the control module, and the input unit is accessible;
a surface of the second side is coupled to a surface of the first side.

17. The electric assist vehicle according to claim 16, wherein:
the control module further comprises a battery compartment and a side cover;
the battery compartment and the side cover are on a third side of the control module, the third side of the control module is opposite to the first side of the control module, the battery compartment is configured to receive the battery, and the side cover rotatably covers the battery compartment.

18. The electric assist vehicle according to claim 17, wherein:
the control module defines an installing hole at a fourth side of the control module, the fourth side is opposite to the second side;
the control module further defines a slot at a surface of the fourth side, the slot communicates with the installing hole, the installing hole is configured to receive the handlebar via the slot.

19. The electric assist vehicle according to claim 14, wherein:
the control unit controls the lighting unit to become a blinking pattern when the control unit of the control module receives a calling signal from an electronic device external to the control module.

20. The electric assist vehicle according to claim 14, wherein:
the handlebar assembly further comprises a monitoring unit, the monitoring unit is arranged on a portion of the handlebar away from the input unit, the monitoring unit is configured to detect heart beat rate data and/or blood oxygen saturation data of a user, the monitoring unit communicates with the control unit of the control module via a wireless connection or a wired connection;
the control unit is configured to obtain the heart beat rate data and/or the blood oxygen saturation data of the user from the monitoring unit, to monitor a health of the user.
